(19) United States
(12) Patent Application Publication
(10) Pub. No.: US 2004/0013742 A1
(43) Pub. Date: Jan. 22, 2004

(54) Biodegradable ceramic for medical use

(75) Inventors: Feng-Huei Lin, Taipei (TW); Guo-Chung Dong, Changhua (TW)

Correspondence Address: BACON & THOMAS, PLLC, 625 SLATERS LANE, FOURTH FLOOR, ALEXANDRIA, VA, 22314

(73) Assignee: Purzer Pharmaceutical Co., Ltd., Taipei (TW)

(21) Appl. No.: 10/434,068

(22) Filed: May 9, 2003

(30) Foreign Application Priority Data
Jul. 17, 2002 (TW) 91115916

(51) Int. Cl. A61K 33/42; C04B 35/00; C09K 3/00

(52) U.S. Cl. 424/602; 106/35; 501/1

(57) ABSTRACT

A biodegradable ceramic for medical use is disclosed, which comprises calcium hydrogenphosphate (CaHPO₄) modified by organic molecules. The organic molecule mentioned above is hexamethylene diisocyanate (HMDI), which is grafted to the calcium hydrogenphosphate through covalent bond. The present invention also relates to a method for preparing the biodegradable ceramics. In addition, the biodegradable ceramic can be used for repair of bone defects. Therefore, the present invention further relates to a method and a device for repair of bone defects, and to a delivery system for the medicine of repair of bone defects.

### BACKGROUND OF THE INVENTION

1. Field of the Invention

The present invention relates to a biodegradable ceramic for medical use and a method for preparing the same, more particularly, to a biodegradable ceramic used for repair of osseous tissue and a method for preparing the same.

The present invention further relates to a method and a device for repair of osseous tissue, and to a delivery system for osseous tissue and osseous tissue healing medicine.

2. Description of Related Art

According to previous studies, neoosteogenesis comes from three sources: the first one is viable osteoblast implantation; the second is osteoconduction, that is, conducting the bone cells around the defects to grow along the implanted scaffold; the third is osteoinduction, that is, mesenchymal cells are induced by certain growth factors to differentiate into osteoblasts and further develop to osseous tissue.

Generally speaking, bone defects are usually repaired by stuffing the cavity with an appropriate substance to maintain the physical status around the defects. The most appropriate material for packing cavities is autogenous graft since it has osteoconduction and osteoinduction properties, but the source and amount of the graft are limited. Recently, many efforts have been made to develop new materials for bone substitutes. Among these, calcium phosphates and bioactive glass have been proven to be biocompatible and bioactive materials that can chemically bond with bone, and have been successfully used clinically for repair of bone defects and augmentation of osseous tissue. However, those bioceramics have only the property of osteoconduction without any osteoinduction. Unlike the autogenous graft, bioceramics do not have a two-phase phenomenon of repair. Said two-phase phenomenon involves direct transfer of viable osteoblasts to the graft and the release of inductive and growth factors from the transplanted bone matrix. Thus, osteoblast further grows into osseous tissue.

Therefore, the approach for a good delivery system of the bioceramics has been a particular focus. Preferably, the bioceramics are also good delivery systems. With the good delivery system in the ceramics, they can receive growth factors from the tissue or additional osseous growth medicine for the osteoinduction.

In addition, for repair of osseous tissue, the biodegradable or bioabsorbable ceramics are preferred because a further operation is not required to remove them once the tissue has been repaired. Monolithic block and disk systems have been developed to the biodegradable delivery system. However, monolithic blocks occlude the bone marrow cavity, which is the most plentiful source of osteoprogenitor cells and thus, the effect of the osseous tissue repair is reduced.

Therefore, a new ceramic material with osteoconduction and osteoinduction properties is needed as a good delivery system. The material must be biodegradable with a particle size that will not occlude bone marrow, and further must be suitable for osteoblast or marrow stromal cell growth. Such a material will not require further operations to remove the bioceramics as the material will employ good bone regenerative characteristics and rapidly achieve bone regeneration and repair.

### SUMMARY OF THE INVENTION

The present invention provides a biodegradable ceramic for medical use, to act as scaffolds of the bone cell, and to have both osteoconduction and osteoinduction properties for osteoblast or marrow stromal cell growth. The material can be made in different shapes and sizes in application for regeneration and repair of various bone defects.

The present invention also provides a biodegradable delivery system; said delivery system is used for regeneration or repair of osseous tissue. Growth or osteoinductive factors of the osseous tissue, and bone healing medicine can all be delivered to the osteoblast or the marrow stromal cell, or the osseous tissue needing to be repaired.

The biodegradable ceramic for medical use in the present invention comprises calcium hydrogenphosphate which is modified by at least one organic molecule. Wherein said organic molecule is hexamethylene diisocyanate (HMDI), which is grafted to said calcium hydrogenphosphate through covalent bond.

The process for preparing a biodegradable ceramic for medical use is described as follows: (A) providing 5 to 20 g of calcium hydrogenphosphate powder with particle size about 0.01 to 1.0 μm; (B) dissolving said calcium hydrogenphosphate powder in an anhydrous organic solvent to form a mixture and stirring said mixture under an anhydrous atmosphere for about 0.5 to 2.0 hour; and (C) adding 3 to 12 ml of hexamethylene diisocyanate (HMDI) to said mixture under said anhydrous atmosphere and keeping the temperature at 20 to 70° C. to stand for about 1 to 6 hours.

Other objects, advantages, and novel features of the invention will become more apparent from the following detailed description when taken in conjunction with the accompanying drawings.

### BRIEF DESCRIPTION OF THE DRAWINGS

FIG. 1 is the thermal gravimetric analysis (TGA) curve and the differential thermal analysis (DTA) curve of calcium hydrogenphosphate (CHP) powder;

FIG. 2 is the thermal gravimetric analysis (TGA) curve and the differential thermal analysis (DTA) curve of surface modified calcium hydrogenphosphate (MCHP);

FIG. 3 is the thermal gravimetric analysis (TGA) curve of surface modified calcium hydrogenphosphate (MCHP) prepared at different temperatures;

FIG. 4 is the differential thermal analysis (DTA) curve of surface modified calcium hydrogenphosphate (MCHP) prepared at different temperatures;

FIG. 5 is the ³¹P-NMR spectrum of surface modified calcium hydrogenphosphate (MCHP);

FIG. 6 is the ¹³C-NMR spectrum of surface modified calcium hydrogenphosphate (MCHP), FIG. 6a is the ¹³C-NMR spectrum of pure HMDI; FIG. 6b is the ¹³C-NMR spectrum of MCHP prepared at 50° C. (Embodiment 4), FIG. 6c is the ¹³C-NMR spectrum of MCHP prepared at 60° C. (Embodiment 5).

### DETAILED DESCRIPTION OF THE PREFERRED EMBODIMENT

Osseous tissue repair comprises three major elements: scaffolds, cells and growth factors. Scaffolds provide an environment for cell attachment, proliferation and differentiation, and maintain the stability of tissue construction. The current applications for tissue engineering mostly are calcium ceramics, more particularly, polyporous hydroxyapatites (HA), which are suitable for the osteoblast growth developed in periosteum and bone marrow cavity. In addition, polyporous hydroxyapatites are biodegradable to perform as the scaffolds for osseous tissue. However, after implanting polyporous hydroxyapatites with osteoconduction ability rather than osteoinduction ability, osteoblast surrounded fails to receive growth factors and to differentiate. Therefore, the surface of polyporous hydroxyapatite has to link with organic groups to become a good delivery system for growth factors. In general, there are two ways for the surface modification of polyporous hydroxyapatites with organic molecules, and one of them is surface adsorption. That is, said organic molecules are adsorbed on the surface of polyporous hydroxyapatites via physical activity. However, with weak adsorption, the organic groups are easily scoured in the physiological environment, and lose their delivery ability. The other way is the covalent binding between said organic group and OH group of the hydroxyapatite.

The present invention uses the second way, utilizing a distinctive organic group to covalently bind the OH group of the hydroxyapatite. The present invention uses the CN group of hexamethylene diisocyanate to covalently bind with the OH group of the hydroxyapatite. For the preparation, 5 to 20 g of calcium hydrogenphosphate powder is dissolved in an anhydrous organic solvent to form a mixture; said organic solvent is preferable dimethylformamide (DMF), dimethyl sulfoxide (DMSO) and chloro carbon (CC). Preferably, said calcium hydrogenphosphate powder is about 0.1 μm. Said mixture is then stirred under an anhydrous atmosphere, such as nitrogen or inert gas atmosphere for about 1 hour. After said calcium hydrogenphosphate powder is completely dissolved in said organic solvent, 5 to 20 g of hexamethylene diisocyanate (HMDI) is added to said mixture under an anhydrous atmosphere, such as nitrogen or inert gas atmosphere, at 20 to 70° C.; preferable is 60° C. and more preferable is 50° C., to react for about 1 to 10 hours; wherein 4 hours is preferred. The appropriate catalyst is added to the reaction if necessary, said preferable catalyst is dibutylin dilaurate. Thus, the surface modified calcium hydrogenphosphate is obtained. The calcium hydrogenphosphate powder is filtrated and washed with DMF to remove the excess HMDI-polymer. Then said surface modified calcium hydrogenphosphate is washed with acetone for three times to remove the residual DMF, and dried.

Due to the binding of said calcium hydrogenphosphate surface with HMDI, the growth factors or osteoinductive factors are delivered to the osteoblast. Thus, another aspect of the present invention is a biodegradable delivery system, comprising said calcium hydrogenphosphate whose surface is modified by HMDI. Said delivery system is used for bone regeneration or repair, and for delivering growth or osteoinductive factors of the osseous tissue, or the additional medicine for bone healing. Preferably, said medicine is growth factors or osteoinductive factors.

While said calcium hydrogenphosphate performs as a good ceramic material for medical use, surface modification by HMDI will further make it become a good delivery system. Thus, another aspect of the present invention is to provide a biodegradable device used for repair of osseous tissue, comprising the above described calcium hydrogenphosphate whose surface is modified by HMDI. A further aspect of the present invention is to provide a method for repair of osseous tissue, comprising administration of calcium hydrogenphosphate whose surface is modified by HMDI to where repair is needed.

Although the present invention has been explained in relation to its preferred embodiment, it is to be understood that many other possible modifications and variations can be made without departing from the spirit and scope of the invention as hereinafter claimed.

A. Preparation and Embodiments

Materials Preparation

Hexamethylene diisocyanate (HMDI) is purchased from Aldrichand used in the experiments without further purification. Calcium hydrogenphosphate (CaHPO₄, CHP) powder is prepared by heating calcium hydrogenphosphate dihydrate (CaHPO₄.2H₂O) at 200° C. for about 8 h, which has been proven as pure CHP both by FTIR and X-ray diffraction (XRD) spectroscopy. Dimethyl formamide (Aldrich, DMF) is purified with distillation and stored over molecular sieves of 4Å. Dibutyltin dilaurate used without purification is purchased from Acros.

### EXAMPLE 1

12.0 g of dried CaHPO₄ powder with an average grain size of about 0.1 μm, 150 ml of DMF, and 0.12 ml of dibutyltin dilaurate are put into a 250 ml flask. In the system, dibutyltin dilaurate is used as a catalyst. The flask is then stirred for 1 h in N₂ atmosphere. 6 ml HMDI is added to the flask subsequently. The reaction is kept at 20° C. under N₂ protection for 4 h to precipitate a surface modified CHP (MCHP). The MCHP powder is filtered and washed with DMF for three times to remove excess HMDI-oligomer. MCHP is then washed with acetone for three times to remove the residual DMF, and dried.

### EXAMPLES 2, 3, 4, 5, AND 6

The reaction process is approximately the same with Example 1 while the only difference is the reaction temperature after adding the HMDI, as follows:

| Example | Reaction temperature |
|---|---|
| 2 | 30° C. |
| 3 | 40° C. |
| 4 | 50° C. |
| 5 | 60° C. |
| 6 | 70° C. |

B. Analysis

### ANALYSIS EXAMPLE 1

### Thermal Analysis

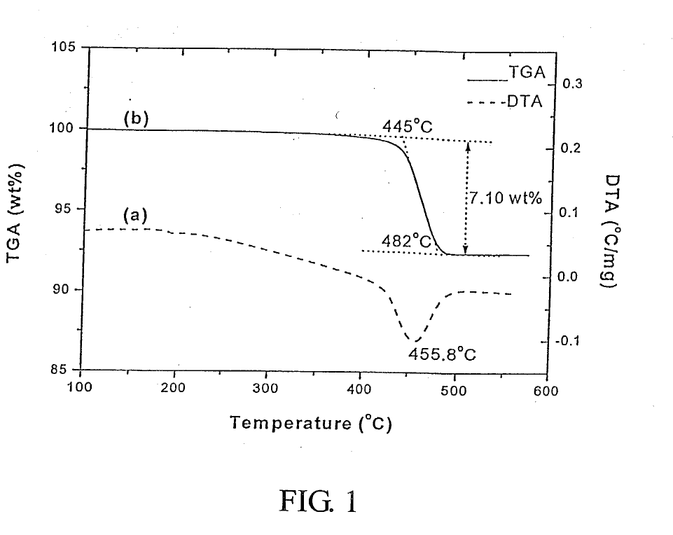

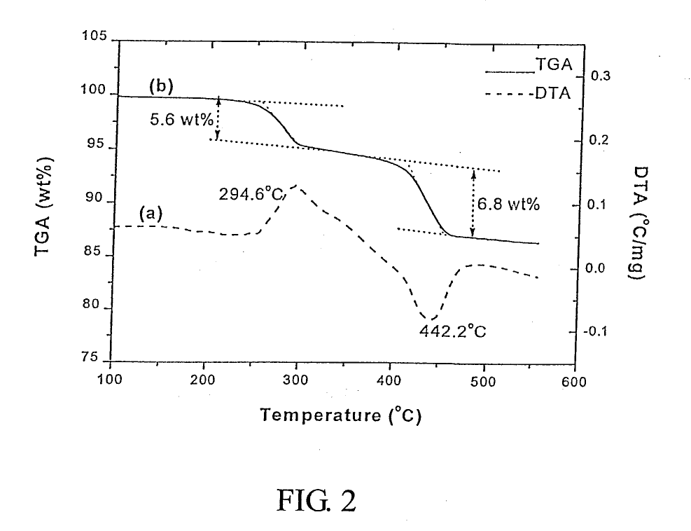

The thermal gravimetric analysis (TGA) and the differential thermal analysis (DTA) are used for thermal analysis data in the present experiment and are performed by a system of SDT 2960 (TA Instruments, Inc., 109 Lukens Drive, New Castle, Del. 19720). In the study, the analysis temperature is from room temperature to 600° C. at a rate 20° C./min. CHP or MCHP powder is put into an alumina crucible for analysis and 10 mg α-Al₂O₃ powder is put into the reference port as reference material. The amount of HMDI grafted on the surface of CHP is supposed to be equal to the weight loss during the heating and it is expressed as weight percentage of the powder's total weight. The results are shown in FIG. 1 and FIG. 2.

Referring to FIG. 1, the thermal gravimetric analysis (TGA) curve and the differential thermal analysis (DTA) curve of calcium hydrogenphosphate (CHP) powder are shown. FIG. 1a (differential thermal analysis; DTA) shows that an endothermic peak appears at the temperature of 455.8° C., which is due to the phase transformation of CHP to dicalcium pyrophosphate (Ca₂P₂O₇). FIG. 1b (thermal gravimetric analysis; TGA) shows an obvious weight loss at the temperature of 445-482° C., which is the H₂O loss during the phase transformation of CHP to Ca₂P₂O₇.

FIG. 2 shows the thermal gravimetric analysis (TGA) curve and the differential thermal analysis (DTA) curve of surface modified calcium hydrogenphosphate (MCHP). FIG. 2a (differential thermal analysis; DTA) shows that there is one exothermic peak and one endothermic peak at the temperature of 294.6° C. and 422.2° C., respectively. In the comparison with FIG. 1a, the endothermic peak at the temperature of 422.2° C. is related with the CHP phase transformation. The exothermic peak at 294.6° C. is due to HMDI burning. FIG. 2b (thermal gravimetric analysis; TGA) shows that there are two weight loss regions. In the comparison with FIG. 1b the weight loss at the second region is resulted from H₂O loss during the CHP phase transformation. At the first region, the weight loss is due to HMDI burning.

From the above, there are two thermal peaks on the DTA pattern and two-stage weight loss on the TGA pattern. The exothermic peak and the first weight loss are due to HMDI burning. The weight HMDI grafted on the surface of CHP is equal to the weight loss in the heating process, thus, the more the weight loss (the difference in the first region) while HMDI burning, the higher the HMDI grafted percentage.

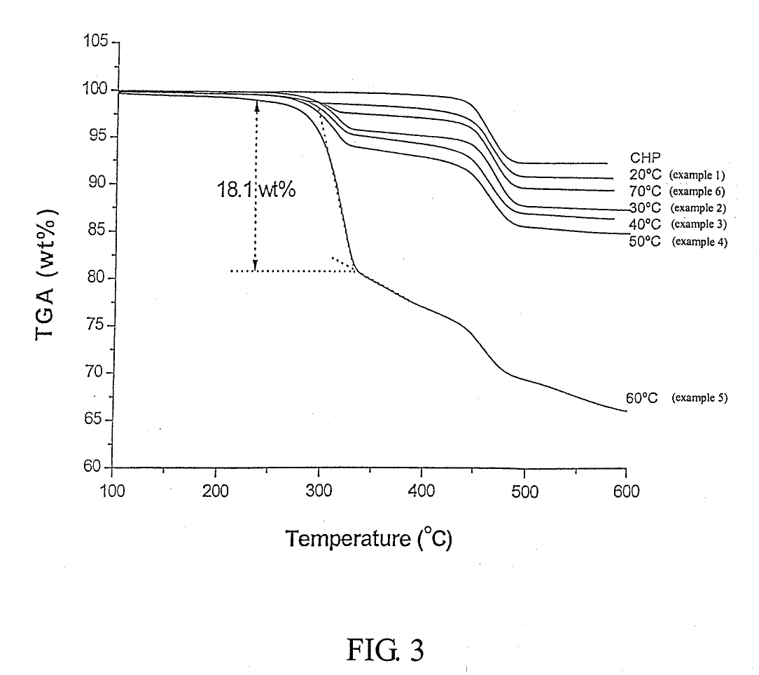

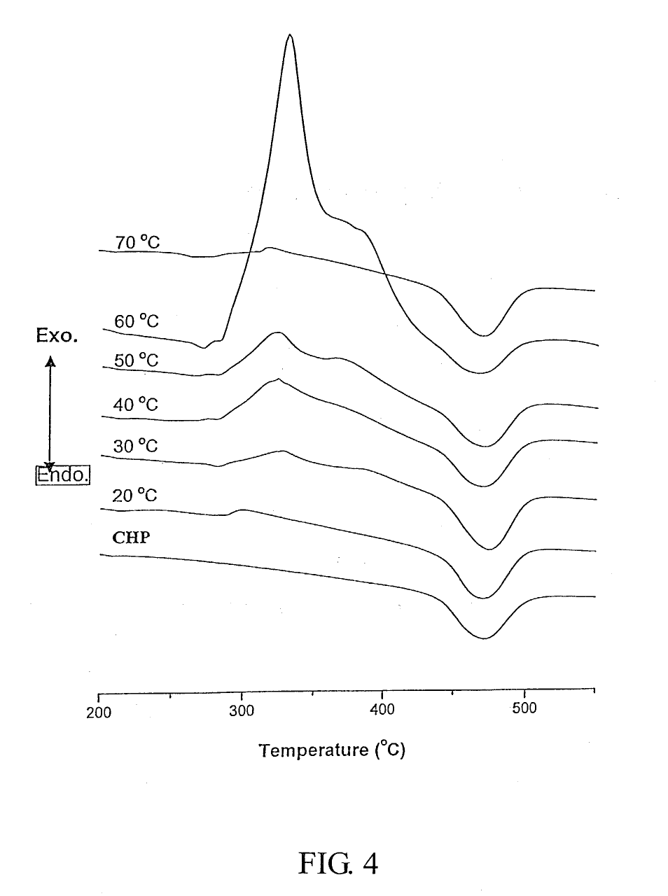

FIG. 3 shows the thermal gravimetric analysis (TGA) curve of the surface modified calcium hydrogenphosphate (MCHP) prepared from each example in the present invention. All the curves have two weight loss regions, that is, all the examples can effectively have HMDI grafted on the surface of CHP. From FIG. 3, MCHP prepared from different temperatures has shown different amounts of surface HMDI. When the reaction temperature rises to 60° C., the greatest amount of HMDI are grafted onto the surface of CHP at around 18.1 wt %. The same result is shown in FIG. 4, which is the differential thermal analysis (DTA) curve of surface modified calcium hydrogenphosphate (MCHP) prepared from each embodiment in the present invention.

From the above, MCHP can be effectively prepared by HMDI modification according to the method in the present invention.

### ANALYSIS EXAMPLE 2

### NMR Analysis

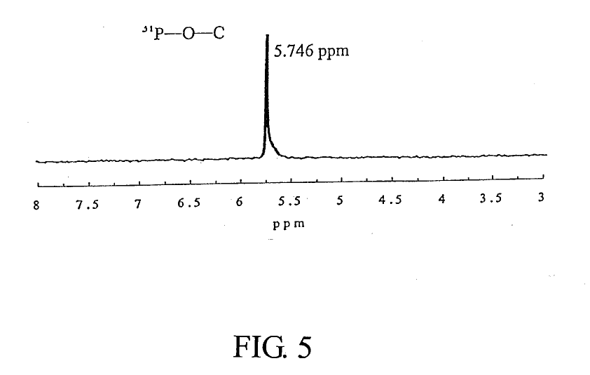

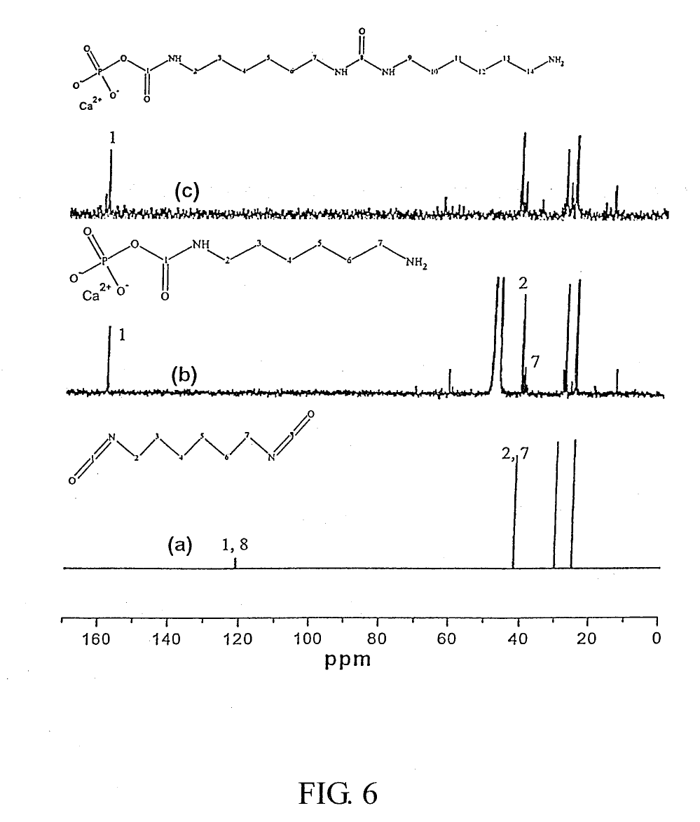

The analysis of MCHP surface linkage comes from ³¹P-NMR spectrum (FIG. 5) and ¹³C-NMR spectrum (FIG. 6). From ³¹P-NMR spectrum (FIG. 5), the P atom in the CHP surface will covalently bind with the C atom in the HMDI via the O atom bridge. That is, the OH group of the phosphate group in the CHP surface will chemically react with CN group in the HMDI to form the covalent bond.

The ¹³C-NMR spectrum in the FIG. 6 shows the surface linkage of CHP with HMDI molecule for MCHP preparation at 50° C. (Example 4) and 60° C. (Example 5). FIG. 6a is the pure HMDI ¹³C-NMR spectrum, FIG. 6b is the regular ¹³C-NMR spectrum and model structure for MCHP prepared at 50° C. (Example 4), and FIG. 6c is the regular ¹³C-NMR spectrum and model structure for MCHP prepared at 60° C. (Example 5). The result in the FIG. 6 is the same with FIG. 5, which proves that the OH group of the phosphate group in the CHP surface will chemically react with the CN group of HMDI to form the covalent bond. In addition, comparing FIG. 6b with FIG. 6c, the surface HMDI molecule can possibly link with other HMDI molecules at 60° C. Thus, the best condition for said reaction temperature is 50° C.

From descriptions mentioned above, in the present invention, calcium hydrogenphosphate (MCHP), whose surface is covalently modified by HMDI, shows good binding ability and efficiency with HMDI. Since CHP possesses biodegradable and bioabsorbable properties, after linkage with organic molecule HMDI, it will become a bone ceramic with good delivery property. Said HMDI molecule can effectively deliver bone growth factors, thus, MCHP comprises both osteoconduction and osteoinduction abilities. Hence, MCHP can be used for the scaffolds of osteoblast and marrow stromal cell development. Moreover, the HMDI molecule can effectively deliver bone growth factors, providing an excellent environment for the osteoprogenitor cells to further differentiate and develop into osseous tissue.

Biomedical Application

Rabbit condyle with 6 mm-diameter defect is implanted by the material prepared from Example 4, and the defect is filled by new osseous tissue and repaired completely after two weeks.

To conclude, the biodegradable ceramic for medical use in the present invention provides the biodegradation property without need for another operation to remove the ceramic material after implantation. The particle size will not occlude bone marrow space, and is suitable for osteoblast or marrow stromal cell development. In addition, the surface HMDI molecule acts as a good delivery system, where bone growth factors can be effectively delivered to the cell. Furthermore, MCHP produces both osteoconduction and osteoinduction abilities. According to the experiment in the present invention, it is proved that the medical biodegradable ceramic can effectively help bone regeneration and repair. According to this property, the biodegradable ceramic for medical use in the present invention can be further used as a method and a biodegradable device for bone repair without need for another operation to remove the ceramic. The bone regenerative ability is fully utilized, and the objects of bone regeneration and repair are thus effectively and rapidly achieved.

Although the present invention has been explained in relation to its preferred embodiment, it is to be understood that many other possible modifications and variations can be made without departing from the scope of the invention as hereinafter claimed.

What is claimed is:

1. A biodegradable ceramic for medical use, comprising calcium hydrogenphosphate which is modified by at least one organic molecule, wherein said organic molecule is hexamethylene diisocyanate.

2. The biodegradable ceramic claimed as claim 1, wherein said organic molecule is grafted to said calcium hydrogenphosphate through covalent bond.

3. A process for preparing a biodegradable ceramic for medical use, comprising the following steps:
(A) providing 5 to 20 g of calcium hydrogenphosphate powder with particle size about 0.01 to 1.0 μm;
(B) dissolving said calcium hydrogenphosphate powder in an anhydrous organic solvent to form a mixture and stirring said mixture under an anhydrous atmosphere for about 0.5 to 2.0 hour; and
(C) adding 3 to 12 ml of hexamethylene diisocyanate to said mixture under said anhydrous atmosphere and keeping the temperature at 20 to 70° C. to stand for about 1 to 6 hours.

4. The process claimed as claim 3, wherein at least one organic solvent in step (B) is selected from the group consisting of dimethylformamide, dimethyl sulfoxide, and chloro carbon.

5. The process claimed as claim 3, wherein said reacting temperature in step (C) ranges from 40 to 60° C.

6. The process claimed as claim 3, wherein said reacting temperature in step (C) is 50° C.

7. The process claimed as claim 3, wherein the step (C) further comprises a step of adding at least one catalyst to said reaction.

8. The process claimed as claim 7, wherein at least one catalyst is dibutylin dilaurate.

9. A biodegradable delivery system, comprising calcium hydrogenphosphate which is modified by at least one organic molecule, wherein said at least one organic molecule is hexamethylene diisocyanate.

10. The biodegradable delivery system claimed as claim 9, wherein said delivery system is used for regeneration or repair of osseous tissue.

11. The biodegradable delivery system claimed as claim 9, wherein said delivery system is used for delivering growth factors or osteoinductive factors of the osseous tissue, and medicine for healing bone defects.

12. The biodegradable delivery system claimed as claim 11, wherein said drug is selected from the group consisting of growth factor and osteoinductive factors.

13. The biodegradable delivery system claimed as claim 12, wherein said at least one organic molecule is grafted to said calcium hydrogenphosphate through covalent bond.

14. A biodegradable device used for repairing bone defects, comprising calcium hydrogenphosphate which is modified by at least one organic molecule, said at least one organic molecule is hexamethylene diisocyanate.

15. The biodegradable device claimed as claim 14, wherein said organic molecule is grafted to said calcium hydrogenphosphate through covalent bond.

16. A method for repairing bone defects, comprising administrating calcium hydrogenphosphate which is modified by at least one organic molecule to a repair site, wherein said at least one organic molecule is hexamethylene diisocyanate.

17. The method claimed as claim 16, wherein said calcium hydrogenphosphate is biodegradable.

18. The method claimed as claim 16, wherein said at least one organic molecule is grafted to said calcium hydrogenphosphate through covalent bond.
